(19) United States
(12) Patent Application Publication
(10) Pub. No.: US 2012/0310309 A1
(43) Pub. Date: Dec. 6, 2012

(54) Adjustable Polarity Laser Device and Polarized Low-Level Laser Therapy Method

(75) Inventors: Steven SHANKS, McKinney, TX (US); Kevin TUCEK, McKinney, TX (US)

(73) Assignee: ERCHONIA CORPORATION, McKinney, TX (US)

(21) Appl. No.: 13/484,922

(22) Filed: May 31, 2012

Related U.S. Application Data
(60) Provisional application No. 61/492,107, filed on Jun. 1, 2011.

(51) Int. Cl. A61N 5/067

(52) U.S. Cl. 607/89

(57) ABSTRACT

An adjustable polarity laser device may treat cancer and other conditions that are responsive to polarized laser energy. The device provides both low-level laser and electrical stimulation of a treatment area, such as a tumor to promote the body's natural defense systems against harmful cells in the treatment area. The device produces low-level laser beams that are polarized with either birefringent or electrically conductive materials disposed in the path of the laser beams. Electrically conductive polarizers are charged by an electric current to impart polarity on the passing laser beams. Treatment methods include applying polarized laser energy to the targeted treatment area. The polarization of each laser beam may be selected and alternated as necessary for the condition being treated. Additionally, photodynamic compounds or photosensitizing agents can be administered to the patient prior to applying polarized laser energy.

### CROSS-REFERENCE TO RELATED APPLICATIONS

This nonprovisional application claims the benefit of co-pending provisional application U.S. Pat. App. Ser. No. 61/492,107 filed on Jun. 1, 2011.

### FIELD OF INVENTION

This invention relates to a device and method for treating cancer and other conditions responsive to polarized laser energy. In particular, this invention relates to the application of selectable polarized laser energy, either alone or combined with a compound having photodynamic properties, to targeted external regions of a patient's body to treat such conditions.

### BACKGROUND

Over the next 20 years, the number of new cancer cases diagnosed annually in the United States are projected to increase by 45 percent, from 1.6 million in 2010 to 2.3 million in 2030, according to a study published Apr. 29, 2009 in the Journal of Clinical Oncology. Additionally, a number of the types of cancers that are expected to increase, such as cancers of the liver, stomach and pancreas, have tremendously high mortality rates. Unless specific prevention or treatment strategies are discovered, cancer death rates also will increase dramatically.

In general, cancer is a class of diseases in which a group of cells display one or more of the following malignant properties: uncontrolled growth (division beyond the normal limits), invasion (intrusion on and destruction of adjacent tissues), and metastasis (spread to other locations in the body via lymph or blood). In contrast, benign tumors are self-limited, do not invade and do not metastasize. Most cancers form a tumor but some, like leukemia, do not.

Cancers are caused by abnormalities in the genetic material of the malignant cells. These abnormalities may be due to the effects of carcinogens, such as tobacco smoke, radiation, chemicals, or infectious agents, or to genetic abnormalities. Cancer-promoting genetic abnormalities may randomly occur through errors in DNA replication or they can be inherited and consequently present in all cells from birth. Cancer research shows that although the malignant cells are abnormal, they maintain certain characteristics of surrounding healthy tissue to a degree that the body's biological defense processes do not identify the malignant cells as harmful or foreign. The body therefore does not resist metastasis of the malignant cells.

Cancer treatment options are varied and numerous. For example, cancer can be treated with surgery, chemotherapy, radiation therapy, or biologic therapy (immunotherapy). Other treatment options include angiogenesis inhibitors therapy, bone marrow transplantation and peripheral blood stem cell transplantation, cryosurgery, gene therapy, proton therapy, and hyperthermia treatment. Additionally, two particular cancer treatment methods take advantage of light therapy: laser therapy and photodynamic therapy. Some of these treatments endeavor to directly destroy malignant cells. Others work to isolate or remove the cells. Still others are directed at changing the cell properties so the body recognizes them as harmful and attacks them naturally.

As to light therapies for treating cancer, ablative lasers are conventionally used to remove cancer or precancerous growths, and non-ablative lasers may be used to relieve symptoms of cancer. Generally lasers are used most often to treat cancers on the surface of the body or the lining of internal organs. For example, lasers can be used to treat basal cell skin cancer and the early stages of cervical, penile, vaginal, vulvar, and non-small cell lung cancer. Additionally, lasers can be used to relieve symptoms of cancer, such as bleeding or obstruction. For example, lasers can shrink or destroy a tumor or polyp blocking a patient's trachea, esophagus, stomach or colon.

In addition to applying laser energy, photosensitizing agents may be applied, which is known as photodynamic therapy. Typically, the photosensitizing agent is injected into a patient's bloodstream and consequently absorbed by cells all over the body. The photosensitizing agent stays in cancer cells longer than it does in normal cells. When tumors are exposed to a specific kind of light, the photosensitizing agent in the tumor absorbs the light and produces an active form of oxygen that destroys nearby cancer cells. The light used for photodynamic therapy can come from a laser or other sources of light.

While two particular cancer treatments have begun to explore the possibilities of light therapy, it would be desirable to develop alternative light therapy methods and devices for treating cancer and other conditions. Generally, light therapy includes light emitted from lasers, light-emitting diodes, broadband bulbs such as xenon lamps and other incandescent bulbs, and other light sources. Light therapy using visible wavelengths has been shown to induce physiological responses in cells via the activation of specific enzymes, becoming an important instrument to treat a wide array of disorders externally.

In particular, low-level laser therapy (LLLT) is an effective method of treating tissues. LLLT utilizes low-level laser energy, that is, the treatment has a dose rate that causes no immediate detectable temperature rise of the treated tissue, no sensation to the patient, and no macroscopically visible changes in tissue structure. Consequently, the treated and surrounding tissue is not heated and is not damaged. LLLT also reduces edema and relieves pain of various etiologies, including successful application post-operatively to liposuction to reduce inflammation and pain. Clinical effects of LLLT include increased protein synthesis, increased bactericidal activity, and activation of cell proliferation, with resultant acceleration of and improved wound healing, improvement of blood microcirculation, regeneration of tissues and improved immunomodulation.

It would be desirable to expand cancer treatment options to include low-level laser therapy. It further would be desirable to use low-level laser therapy devices to aid biological processes of identifying and combating cancer cells in the body. Therefore, an object of this invention is to provide a low-level laser device with varied and adjustable polarity and to provide a non-invasive method of treating cancer and other conditions with polarized low-level laser therapy.

### SUMMARY OF THE INVENTION

This invention is an adjustable polarity laser device and method for treating conditions such as cancer that are responsive to polarized laser energy. The device is a laser device that can simultaneously provide multiple types of low-level laser therapy treatments. The device enables laser light of different pulse widths, different beam shapes and spot sizes, and different polarities to be applied externally to a patient's body. The device includes two or more laser beams produced by one or more laser sources. The laser beams are polarized with polarizers disposed in the path of the laser beams. In the preferred embodiment, two separate laser beams are produced simultaneously, one laser beam being charged with a first polarity by a first polarizer and the other producing a beam charged with a second polarity by a second polarizer. In the preferred embodiment these polarizers are comprised of electrically conductive metal. Preferably each laser beam is emitted from its own semiconductor diode laser source.

The method for treating conditions responsive to polarized laser energy comprises positioning a laser device having at least one laser energy source and at least one arrangement of polarity control elements so that polarized laser energy is applied to a targeted treatment area. The polarization of each laser beam from each laser source is selected and alternated as necessary for the condition being treated. Additionally, photodynamic compounds or photosensitizing agents can be administered to the patient prior to applying polarized laser energy. The compounds or agents can be applied topically, ingested, injected at the targeted area, or injected into the patient's blood stream. Treatments can be repeated over a period of hours, days or weeks, and additional targeted areas can be treated.

### BRIEF DESCRIPTION OF THE DRAWINGS

FIG. 1 illustrates the application of low-level laser radiation to a targeted area of cancerous cells.

FIG. 2 illustrates the application of low-level laser radiation to a targeted area of cancerous cells with a laser device capable of producing at least two laser beams.

FIG. 3 illustrates the application of low-level laser radiation to a targeted area of cancerous cells with a laser device capable of producing at least three laser beams.

FIG. 4 is an electrical schematic illustration of a preferred embodiment of the present invention.

FIG. 5 is a perspective illustration of two lasers and polarity control elements according to the present invention.

FIG. 6 is a perspective illustration of a single laser and polarity control elements according to an alternative embodiment of the present invention.

FIG. 7 is a schematic illustration of a laser, polarity control element, and optical arrangement for applying laser light according to the preferred embodiment of the present invention.

FIG. 8 is a schematic illustration of a laser, polarity control element, and alternative optical arrangement for applying laser light according to the present invention.

FIG. 9 is an electrical schematic illustration of an alternative embodiment of the present invention.

### DETAILED DESCRIPTION OF THE INVENTION

The present invention is an adjustable polarity laser device and method of treating conditions, such as cancer, with polarized laser energy. The method combines low-level laser therapy with polarization selection and control. Two types of polarity may be exhibited in the device and method. The term “electrical polarity” conventionally refers to the direction of the flow of electrons from one pole to another, specifically from the negative pole to the positive pole. Electrical polarity exhibits a positive and a negative polarity. The term “laser polarity” conventionally refers to the orientation of the oscillation of the laser beam's electric field with respect to the propagation direction of the laser beam. Laser polarity exhibits more complicated polarity, in which the oscillating electric fields can have longitudinal as well as transverse components. The laser beams of the present device induce an electrical current in tissue impinged by the beams, by either the electrical polarity or laser polarity, or both. As used herein, the terms “polarity” and “polarize” used with reference to a laser beam describes either the electrical or laser polarity of the beam, or both, unless “electrical polarity” or “laser polarity” is specifically stated.

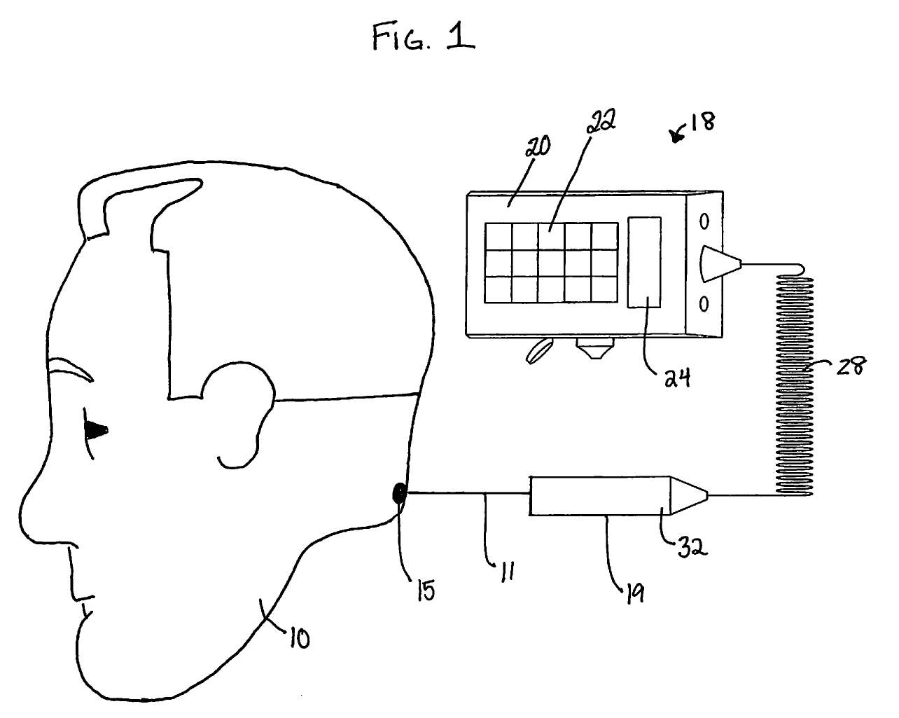

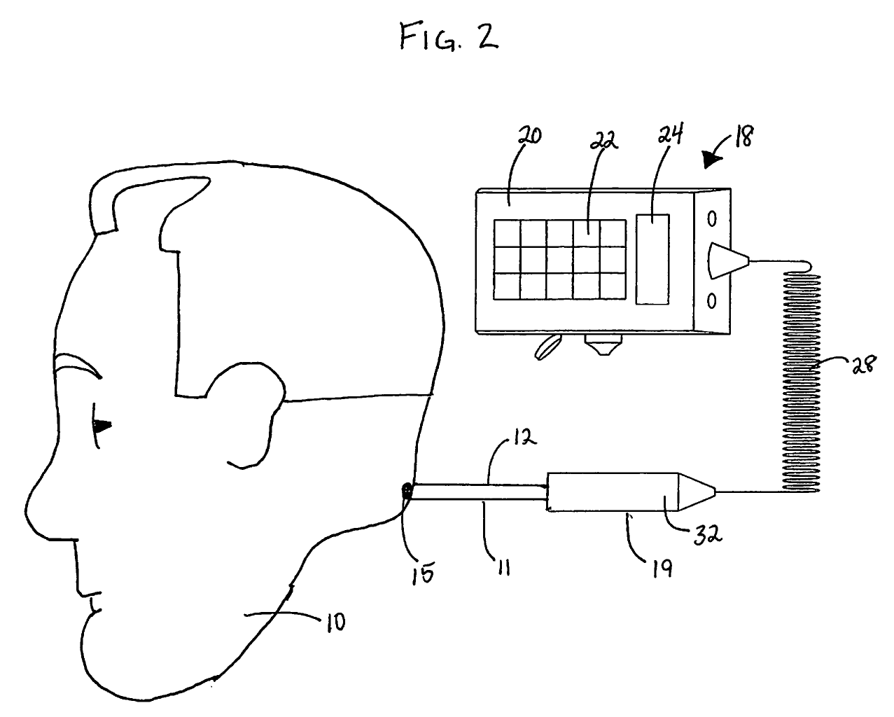

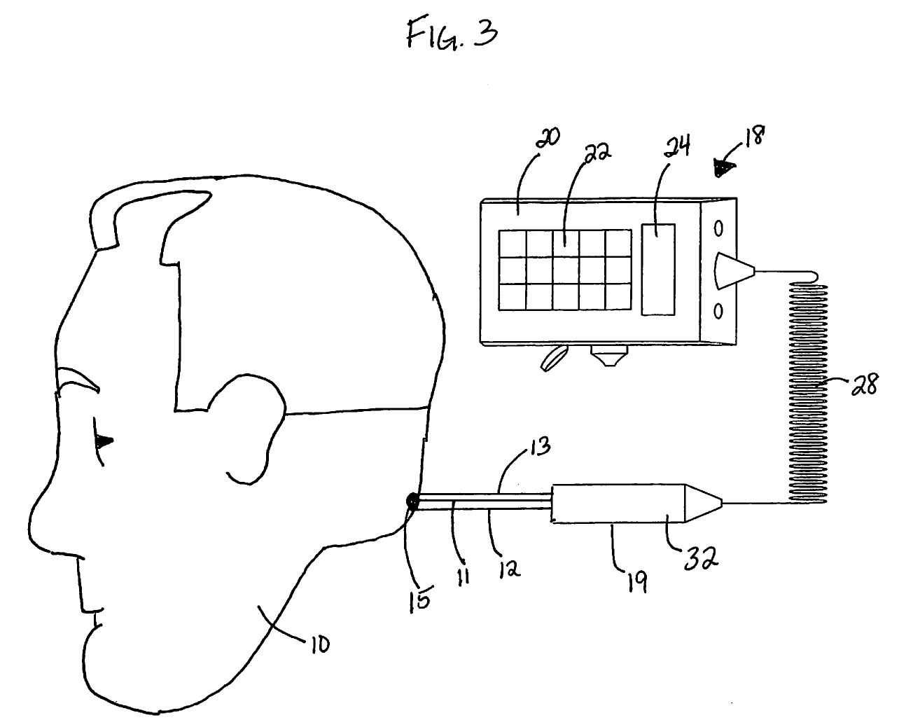

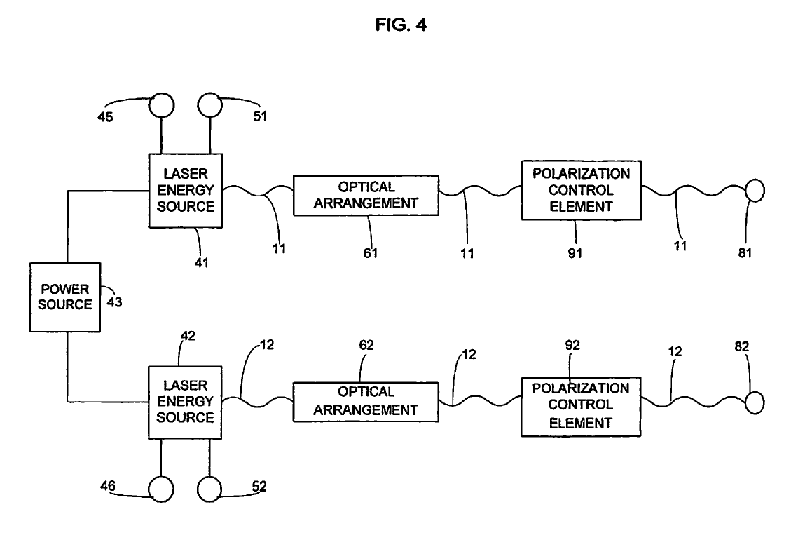

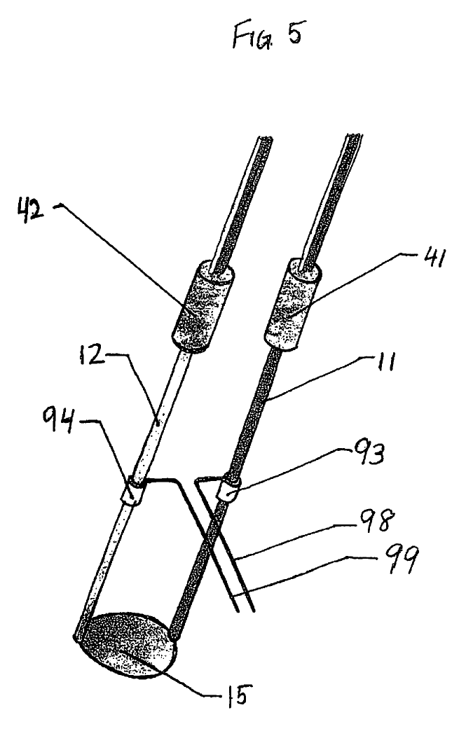

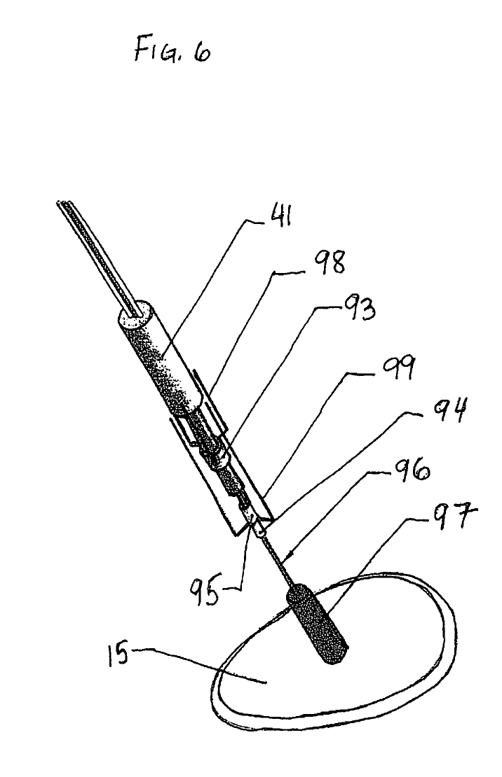

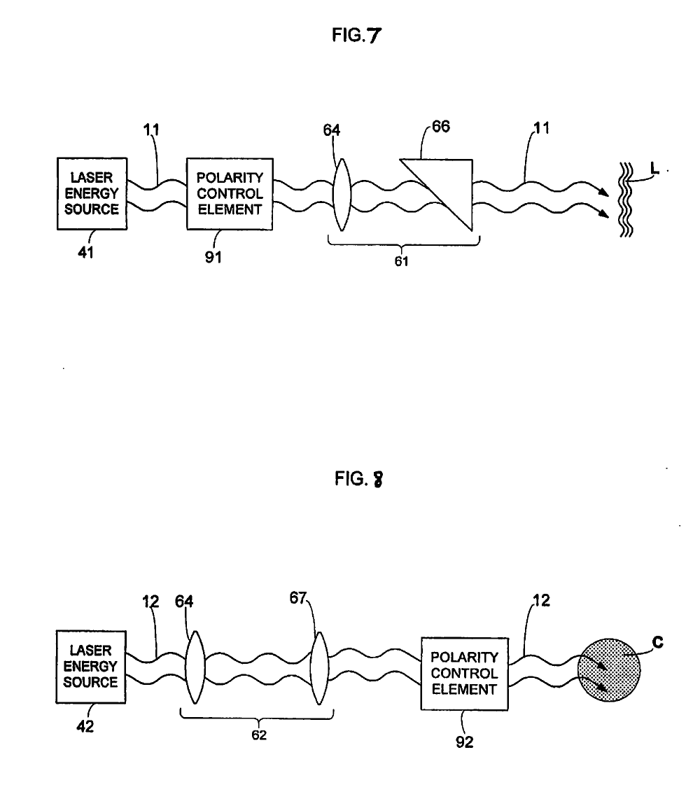

Referring to FIGS. 1-6, the laser device projects one or more laser beams onto human tissue, and further imparts a different electrical charge on each laser beam such that a complete electrical circuit is formed between the laser beams, the human tissue, and one or more power supplies in the laser device. The laser device may include one laser beam 11 having adjustable polarity, as shown in FIGS. 1 and 6, or a plurality of laser beams, such as the two- and three-beam embodiments as shown in FIGS. 2-5, each laser beam having selectable uniform or varied polarity. A laser beam has uniform polarity when the beam has an unchanging polarity within the beam's cross-section. In contrast, a laser beam has varied polarity when the beam changes polarity at least once within its cross-sectional area. The polarity may be further varied by the device, in a switched or pulsed polarity as described below.

Each laser beam is generated by a laser energy source. Laser energy sources known in the art for use in low-level laser therapy include plasma tube lasers; Helium-Neon lasers, particularly those having a 632 nm wavelength; and semiconductor diode lasers with a broad range of wavelengths between 405-1500 nm. Diode lasers at 405 nm, 510 nm, 625 nm, 633 nm, 635 nm, 670 nm and 1064 nm (infrared) have been shown to work with varying degrees of success. Solid state and tunable semiconductor laser diodes may also be employed to achieve the desired wavelength.

The laser energy sources in the preferred embodiment are two semiconductor laser diodes that produce light in the red range of the visible spectrum, having wavelengths of about 635 nm. Other suitable wavelengths are used for other particular applications. While some LLLT regimens include invisible laser light, it is advantageous to utilize at least one laser beam in the visible energy spectrum so that the operator or practitioner can see the laser light as it impinges the patient's body and the area treated can be easily defined. Preferably, each laser beam has its own discrete laser energy source, but alternatively a single laser energy source may generate a beam that is then split using a refractive element to produce two or more beams. The refractive element may have birefringent properties which imparts a different laser polarity upon each of the resulting laser beams.

Different therapy regimens require diodes of different wattages and wavelengths. The preferred laser diodes use less than one watt of power each. Diodes of various other wattages may also be employed to achieve the desired laser energy for the given regimen.

The operational parameters of the device are controlled as needed to perform one or more of the treatment methods described below, and further may also be controlled to perform other LLLT treatments known in the art. As shown in FIGS. 1-3, the laser device 18 may have a user input 22 and display 24, a housing 20 in which a power supply, control circuitry, and additional laser device components are housed; a wand 19 having a housing 32 in which laser polarity control elements and optical components are housed; one or more laser beams 11, 12, 13 exiting the wand 19; and a cord 28 for connecting the wand 19 to the device 18. The laser beams 11, 12, 13 impinge the targeted area 15 on the patient 10 for treatment.

FIG. 4 further illustrates the laser device 18 of the preferred embodiment. As shown, a first laser source 41 and a second laser source 42 are connected to a power source 43. The power source preferably provides direct current, such as that provided by a battery, but may instead provide alternating current such as that provided by conventional building current that is then converted to direct current. Separate control means 45, 46 are connected to the laser energy sources 41, 42 respectively and act as on/off switches to control the period of time the laser light is generated. These laser energy sources can be energized independently or simultaneously which, throughout this specification, refers to acts occurring at generally the same time.

Control means 51, 52 are connected to the laser energy sources 41, 42, respectively, to form control circuits that control the duration of each pulse of laser light emitted, referred to herein as the pulse width. When there are no pulses, a continuous beam of laser light is generated. Pulse widths of different lengths may be employed to achieve the desired effect on the patient's tissue, ranging from a constant wave (infinite pulse width) to any shorter pulse width. The goal of LLLT regimen is to deliver laser energy to the targeted tissue utilizing a pulse width short enough to sufficiently energize the targeted tissue and avoid thermal damage to adjacent tissue.

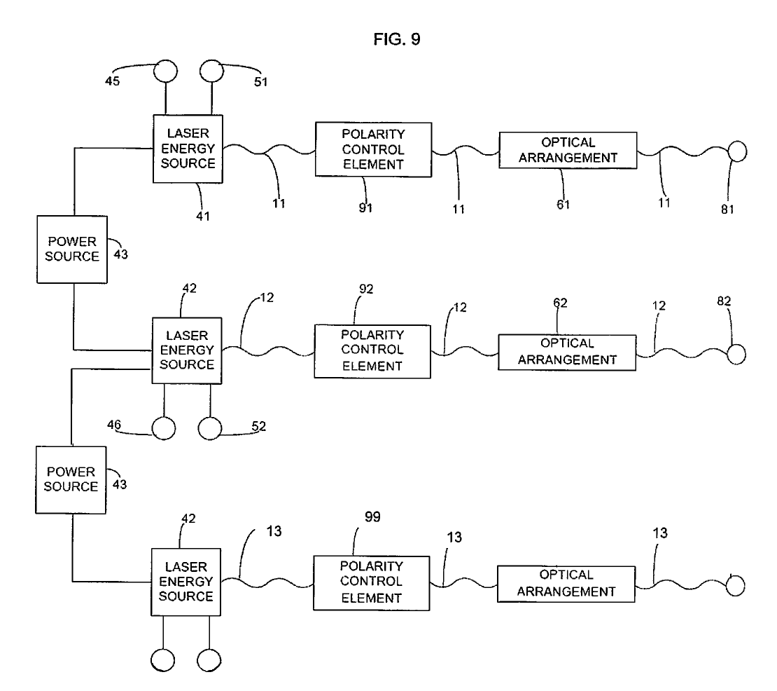

Each laser beam 11, 12 exits the laser and is shown through polarity control elements 91, 92. Optionally, each laser beam 11, 12 may also pass through optical arrangements 61, 62, respectively, to produce beam spots 81, 82, respectively, of certain shapes. The beam spot is the cross-sectional shape and size of the emitted beam as it exits the optical arrangement. For example, a laser beam of circular cross-section creates a circular beam spot as the laser light impinges the patient's skin. If the laser light is emitted in the visible range, a circular spot can be seen on the patient's skin of substantially the same diameter as the laser beam emitted from the optics arrangement. The optical arrangements can be positioned in the beam path either before the polarity control elements, as shown in FIGS. 4 and 8, or after the polarity control elements, as shown in FIGS. 7 and 9.

The polarity control elements 91 and 92 impart a desired polarity on the laser beams 11 and 12, respectively. The polarity control elements 91, 92 comprise at least polarizers 93 and 94 (see FIG. 5) made of any electrically conductive metal such as grade 303 stainless steel. A current is applied to the polarizers 93, 94 to positively or negatively charge. The laser beams 11, 12 each contact, pass along, or pass through apertures in the polarizers 93, 94. The charge on the polarizer 93 determines the polarity of the exiting laser beam 11 as described below. Each of the polarity control elements 91 and 92 may carry a constant or pulsed charge and can produce a fixed polarity laser beam, or a laser beam with a changeable or alternating polarity.

The polarity of the first laser beam 11 cooperates with the polarity of the second laser beam 12 to induce the current in the target area 15. Either the electrical polarity or the laser polarity of the first laser cooperates with the electrical polarity or the laser polarity of the second laser. Alternatively, both the electrical polarity and the laser polarity of the first laser cooperate with the electrical polarity and the laser polarity of the second laser.

An electrical polarity may be imparted on each laser beam 11, 12. The polarizers 93, 94 attract or repel electrons when the current is applied, and these electrons may be carried by the laser beams 11, 12 to or from the targeted area 15. In this sense, the device may cause the laser beams 11 and 12 to become electrically charged so that, at the targeted area 15, one laser beam acts as an anode and the other laser beam acts as a cathode when the polarizers 93, 94—and thus the laser beams 11, 12—carry opposite charges. The electrons in the cancer cells are thus repelled by one beam and attracted to the other, causing an electric current on or through the cancel cells and ionizing them. Once ionized, the cancer cells may be recognized by the patient's 10 body as foreign or harmful and be attacked by the body's natural defenses.

It is further believed that the electrical charge of the polarizers 93, 94 may orient the electric field of the laser beams 11, 12 according to the charge applied. That is, the specific laser polarity of the beam 11 depends on whether the polarizer 93 is positively or negatively charged. Thus, similar to the ionization created by the electrical polarity as explained above, when laser beams 11, 12 having opposite laser polarities contact the targeted area 15, the electric fields of the beams 11, 12 cooperate to draw the electrons in the cancer cells toward one of the beams, causing an electric current and ionizing the cancer cells. Once ionized, the cells may be identified by the patient's 10 body as foreign or harmful be attacked by the body's natural defenses.

FIGS. 5 and 6 illustrate in detail the polarity control elements 91 and 92 of the present invention. FIG. 5 illustrates a dual diode laser arrangement where there are two laser sources 41 and 42 emitting two laser beams 11 and 12 respectively. Laser beams 11 and 12 can be the same wavelength and color or can be two different wavelengths and colors. There are also two polarizers 93 and 94 respectively. As shown, polarizers 93 and 94 are each a hollow tube made of electrically conductive metal. Preferably the tubes are sized so that the outer edge of beam 11 comes in contact with polarizer 93, and the outer edge of beam 12 comes in contact with polarizer 94. Alternatively, the laser beams 11, 12 pass through the aperture in the polarizer and do not touch the tube. Control elements (not shown) control the charge delivered through wires 98 and 99. Polarizer 93 is positively or negatively charged through wire 98, and polarizer 94 is positively or negatively charged through wire 99. The polarizer itself is charged, and an electric field spans any aperture therein. Therefore, by controlling and adjusting polarizers 93 and 94, a user can control the polarity of the emitted laser beam and can alternate or change the polarity between a first polarity and a second polarity as desired. With the present invention, the polarity of each laser beam can be either a substantially continuous first polarity, or it can alternate between a first and second polarity. The rate of alternating the polarity also can be selected and controlled with the control elements.

Referring to FIG. 6, for a single diode laser arrangement where there is one laser source 41 and one initial beam 11, the polarity control elements include two polarizers 93 and 94 and an insulator 95. As with the embodiment detailed above, polarizers 93 and 94 are charged to a first or second polarity through wires 98 and 99 respectively. Polarizers 93 and 94 are also hollow tubes as described above. The outer edge of beam 11 comes into contact with polarizer 93. Then beam 11 is split into an inner beam 96 and outer beam 97 by insulator 95. Inner beam 96 then comes into contact with polarizer 94. With this arrangement a single diode laser can produce a beam that is both positively charged and negatively charged. As with the dual diode arrangement, polarizers 93 and 94 allow a user to control and alternate the polarity of the inner and outer beams between a first and second polarity as desired. The polarity of the beams can be either a substantially continuous first polarity or second polarity, or can alternate between a first and second polarity. The rate of alternating the polarity also can be selected and controlled with the control elements. The inner and outer beams can be of an opposite polarity, and each can have a fixed polarity or can alternate polarity.

FIG. 7 further illustrates a laser device with a polarity control element 91 for polarizing the emitted laser beam 11 and an optical arrangement 61 for shaping the beam 11 and creating a linear shape L. The optical arrangement has a collimating lens 64 and a line generating prism 66 disposed in serial relation to the laser energy source 41. The collimating lens 64 and line generating prism 66 receive and transform the generated beam of laser light into a line of laser light L. As an alternative, a suitable electrical or mechanical arrangement or combination thereof could be substituted for the optical arrangement to achieve a desired spot shape. As shown in FIG. 7, the polarity control element 91 is positioned in the beam path between the laser energy source 41 and the optical arrangement 61. Alternatively, the optical arrangement 61 can be positioned between the laser energy source 41 and the polarity control element 91, or the optical arrangement 61 can be omitted altogether.

As shown in FIG. 8, the second optical arrangement 62 of the device can alternatively include a collimating lens 64 and a beam spot shaping lens 67. As with the first optical arrangement 61, the collimating lens 64 and beam spot shaping lens 67 receive and transform the generated beam of laser light 12 into a circular beam spot of laser light C. As an alternative, a suitable electrical or mechanical arrangement or combination thereof could be substituted for the optical arrangement to achieve a desired spot shape. As shown in FIG. 8, the optical arrangement 62 is positioned in the beam path between the laser energy source 42 and the polarity control element 92. Alternatively, the polarity control element 92 can be positioned between the laser source 42 and the optical arrangement 62, or the optical arrangement 62 can be omitted altogether.

Referring to FIG. 9, the device may have an odd-numbered plurality of laser energy sources 41, 42, 50 that are powered by two power sources 43, 53 having opposite electrical polarity. In order to complete the electrical circuits for each power source 43, 53, one of the laser energy sources 42 serves as the “middle” source and is connected to both power sources 43, 53. The other laser energy sources 41, 50 are split evenly between the power sources 43, 53. The polarity control element 99 for the middle source 42 polarizes the middle beam 12 opposite those of the other beams 11, 13. The current induced in the targeted area 15 flows toward or away from the beam spot of the middle beam 12, allowing the middle beam 12 to complete the circuits for both power sources 43, 53.

The device may utilize as many lasers and polarity control elements and optical arrangements as necessary to obtain the desired emissions, polarity, and spot shapes. For example, the device may employ two laser diodes each with a collimating lens, such that two substantially circular spot shapes are achieved. Alternatively, for example, the device may employ two laser diodes each with an optical arrangement such that two substantially linear spot shapes are achieved. In another example, more than two lasers may be used and optical arrangements aligned such that two or more of the laser beams have substantially similar spot shapes. Additionally, for each of the elements, the polarity can be controlled independently such that two laser beams of identical polarity are achieved, two laser beams of opposite polarity are achieved, or multiple laser beams of various polarities are achieved. In any embodiment, the beams impinge the targeted area 15 in proximity to each other in order to induce the current, which is typically extremely small, between the beams of opposite polarity. The beam spots are not co-incident, which would cause the circuit to be completed without inducing current and ionizing the cells.

As discussed previously, laser beams 11 and 12 exit wand 19. Wand 19 is preferably a lightweight, handheld pointer. Wand 19 is preferably an elongated hollow tube defining an interior cavity which is shaped to be easily retained in a user's hand. In the preferred embodiment, the laser energy sources 41, 42 are mounted in the wand's interior cavity, although the laser energy sources could be remotely located and the laser light conducted by fiber optics to the wand. The polarity control elements and optical elements are housed in the wand as well. The wand may take on any shape that enables the laser light to be directed as needed such as tubular, T-shaped, substantially spherical, or rectangular.

FIGS. 1, 2, and 3 illustrate the laser device of the present invention as it is used to treat cancer and other conditions responsive to polarized LLLT. Preferably, to treat cancer and other conditions, the targeted area 15 is treated by applying energy of different or alternating polarity. FIG. 1 illustrates a laser device 18 having a single laser beam 11 that exits wand 19. Laser beam 11 can be positioned on targeted area 15, and the polarity of laser beam 11 can be periodically switched from a first polarity to a second polarity at a constant or varying rate. Additionally, the laser pulse rate and pulse width can be controlled as well as the treatment duration.

FIG. 2 illustrates an alternative embodiment of laser device 18 having two laser beams 11 and 12 that exit from wand 19. For this embodiment, the two laser beams can be positioned on opposing sides or areas of targeted area 15. Each beam can be controlled independently so that one beam applies laser energy having a first polarity while the other beam applies laser energy having a second polarity. Alternatively, both beams can be adjusted so they apply uniform polarity that is periodically switched from a first polarity to a second polarity, or both beams can independently switch between first and second polarities at different rates. Additionally, other characteristics of each laser beam can differ or be independently controlled. For example, each laser beam can have a different wavelength or a different spot shape. Additionally, the laser pulse rate and pulse width can be controlled as well as the treatment duration.

Additionally, laser energy can further be applied to the targeted area using three or more laser beams. Each beam can be controlled independently so that each beam applies laser energy of a constant polarity or of alternating polarity according to treatment protocols. For example, each laser beam can be any polarity so that two or more laser beams can have the same polarity or different polarities. Additionally, other characteristics of each laser beam can differ or be independently controlled. For example, each laser beam can have a different wavelength or a different spot shape. Additionally, the laser pulse rate and pulse width can be controlled as well as the treatment duration.

In addition to applying polarized laser energy, the targeted area 15 can be treated with a photosensitizing agent or a compound having photodynamic properties in order to enhance the effects of the polarized laser energy treatment. A compound with photodynamic properties will attach to the cancerous cells, for example, which will then respond to the polarized laser energy in such a way that the tumor is destroyed. The agent or compound can be applied topically, ingested, injected at the targeted area 15 or injected into the patient's bloodstream.

In general, there are a number of variables in determining sufficient and appropriate laser therapy for this application. The variables include the wavelength of the laser beam, the area impinged by the laser beam, laser energy, pulse frequency, pulse width, polarity of the laser beam, polarity pulse rate, treatment duration, depth and type of cancer or condition being treated, and tissue characteristics such as the thickness of the patient's skin, thickness of fatty tissue, and other biological factors peculiar to each patient. The wavelength of the applied laser energy depends on the nature of the condition or cancer being treated, among other factors, and ranges from ultraviolet to infrared. Preferably, however, the applied laser energy is in the visible spectrum, from about 396 nm to about 800 nm. Pulse frequencies from 0 to 100,000 Hz or more may be employed to achieve the desired effect on the patient's condition. When there are no pulses, a continuous beam of laser light is generated. The patient feels no sensation of the low-level laser energy being applied.

The preferred treatment is to apply laser energy to the targeted area two times a week for about two weeks. Alternatively, laser energy can be applied repeatedly over longer or shorter time periods, such as repeated treatments within forty-eight hours. Additionally, the targeted area 15 can be treated one or more additional times during a single treatment and additionally over a period of days or weeks. The procedure may be repeated in one or more additional areas to treat additional cancerous locations. Additional laser energy may be applied over the entire extremity or larger area surrounding targeted area 15 or over the entire body of the patient to stimulate other body systems such as the lymphatic, circulatory, and nervous systems.

Applying polarized low-level laser energy causes no immediate detectable temperature rise of the treated tissue and no macroscopically visible changes in tissue structure. Low-level laser energy penetrates the skin and is specific to the depth of the desired zone of the tumor or other condition to be treated. Consequently, the treated and surrounding tissue is not heated and is not damaged. Preferably the laser light is visible to the human eye so that the area of application is easily determined. The laser energy is optionally applied by a scanning laser or by a non-scanning laser freely moved by a practitioner in a predetermined manner. Alternatively, the laser energy can be emitted from a stationary source, such as an arm that emits laser energy that is attached to a wall or a stand.

Following are some examples of treating a targeted area of a patient's body having cancerous cells or other conditions responsive to polarity therapy:

### Example 1

The targeted area for polarity therapy is the back of a patient's neck. A photodynamic compound is applied topically to the targeted area. The patient's neck is then each treated with polarized laser energy, using a single 635 nm semiconductor diode laser at a power of 10 mW. The laser device includes an insulator that divides the laser beam into an inner beam and an outer beam. Initially, one polarizer imparts a positive electrical polarity on the inner beam and a second polarizer imparts a negative electrical polarity on the outer beam. The laser energy is applied for 20 minutes in a back-and-forth sweeping motion across the patient's neck without touching the patient. During the treatment the polarity alternates between first and second polarities. Treatment is repeated two more times over a two week period.

### Example 2

The targeted area for polarity therapy is the back of a patient's neck. The patient's neck is then each treated with polarized laser energy using a first 635 nm semiconductor diode laser at a power of 10 mW and a second 635 nm semiconductor diode laser at a power of 10 mW. The laser beam from the first semiconductor diode laser is positioned near one side of the targeted area and the laser beam from the second semiconductor diode laser is positioned near an opposing side of the targeted area. The laser energy is applied for 20 minutes without touching the patient. During the treatment a first polarizer imparts a first polarity on the laser beam emitted from the first semiconductor laser, and a second polarizer imparts a second polarity on the laser beam emitted from the second semiconductor laser. The first polarity includes laser polarity that is circular with clockwise chirality, and the second polarity includes laser polarity that is circular with anti-clockwise chirality. Treatment is repeated two more times over a two week period.

### Example 3

The treatment of Example 2 is used, except the first polarity includes laser polarity that is linear with vertical orientation, and the second polarity includes laser polarity that is linear with horizontal orientation.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

We claim:

1. A laser device comprising:
a. at least one laser energy source configured to generate a low-level laser beam;
b. at least one polarizer positioned so that the outer edge of the laser beam contacts the polarizer to polarize the laser beam.

2. The laser device of claim 1 wherein the polarizer comprises a birefringent material.

3. The laser device of claim 1 wherein the polarizer comprises a conductive material.

4. The laser device of claim 3 wherein the polarizer is charged by an electric current to polarize the laser beam.

5. The laser device of claim 4 further comprising an insulator positioned in the path of the laser beam to split the laser beam into an inner beam and an outer beam, each having an outer edge, wherein one polarizer contacts the outer edge of the outer beam and the other polarizer contacts the outer edge of the inner beam, and wherein the polarizers are oppositely charged by the electric current.

6. The laser device of claim 4 wherein the laser energy sources comprise a first laser energy source configured to generate a first laser beam and a second laser energy source configured to generate a second laser beam.

7. The laser device of claim 6 wherein the polarizers comprise a first polarizer positioned so that the outer edge of the first laser beam contacts the first polarizer, and a second polarizer positioned so that the outer edge of the second laser beam contacts the second polarizer, the first and second polarizers being oppositely charged by the electric current.

8. The laser device of claim 7 wherein each polarizer comprises a ring through which one of the laser beams passes.

9. The laser device of claim 8 wherein the ring is stainless steel.

10. The laser device of claim 7 wherein:
a. the laser energy sources further comprise a third laser energy source configured to generate a third laser beam;
b. the polarizers comprise a third polarizer positioned so that the outer edge of the third laser beam contacts the third polarizer; and
c. the third polarizer is charged oppositely to either the first or second polarizer by the electric current.

11. The laser device of claim 7 further comprising control means for activating the first and second laser energy sources and applying a current to one or both of the first and second polarizers in order to produce oppositely-polarized first and second laser beams.

12. A low-level laser therapy device comprising:
a. a first power source;
b. a first laser energy source electrically connected to the first power source and configured to generate a low-level first laser beam having a circular cross-section;
c. a second laser energy source electrically connected to the first power source and configured to generate a low-level second laser beam having a circular cross-section;
d. a first polarizer electrically connected to the first power source, the first polarizer comprising a stainless steel ring having an inner diameter equal to the diameter of the first laser beam, the first polarizer being positioned so that the first laser beam passes through it;
e. a second polarizer electrically connected to the first power source, the second polarizer comprising a stainless steel ring having an inner diameter equal to the diameter of the second laser beam, the second polarizer being positioned so that the second laser beam passes through it; and
f. control means for activating the first and second laser energy sources and applying a current to one or both of the first and second polarizers in order to produce oppositely-polarized first and second laser beams.

13. A laser device for treating a targeted area on a patient, the device comprising:
a. at least one laser energy source configured to generate a low-level laser beam; and
b. at least one polarizer positioned between the laser source and the targeted area on the patient.

14. A method of applying low level laser therapy to a treatment area on a patient's skin, the method comprising directing one or more polarized low-level laser beams to the treatment area so that a current is induced through the patient's skin.

15. The method of claim 14 wherein two laser beams are directed to the treatment area, the laser beams having opposite polarity.

16. The method of claim 14 wherein the laser beams have different wavelengths.

17. The method of claim 16 wherein at least one of the laser beams has a wavelength of 635 nm.

18. The method of claim 14 wherein one laser beam is directed to the treatment area, the laser beam having an outer beam and an inner beam of opposite polarities.

19. The method of claim 18 where the laser beam is generated by a laser device comprising:
a. a power source; a laser energy source electrically connected to the power source and configured to generate the laser beam;
b. a first polarizer electrically connected to the power source and disposed in the path of the laser beam such that the laser beam contacts the first polarizer at the laser beam's outer edge;
c. an insulator disposed in the path of the laser beam and configured to split the laser beam into an outer beam and an inner beam; and
d. a second polarizer electrically connected to the power source and disposed in the path of the inner beam.

20. The method of claim 14 wherein three laser beams are directed to the target area, the laser beams comprising a left beam and a right beam of the same polarity and a middle beam of the opposite polarity.
